(19) United States
(12) Patent Application Publication
(10) Pub. No.: US 2023/0358749 A1
(43) Pub. Date: Nov. 9, 2023

(54) EARLY CANCER DIAGNOSIS METHOD AND DIAGNOSTIC KIT USING INTERFERON GAMMA GENE CONCENTRATION MEASUREMENT IN EXOSOMES

(71) Applicant: DIOGENE CO., LTD., Gyeonggi-do (KR)

(72) Inventors: Seong Sik LIM, Gyeonggi-do (KR); Yeon Soo KIM, Gyeonggi-do (KR)

(21) Appl. No.: 17/886,778

(22) Filed: Aug. 12, 2022

(30) Foreign Application Priority Data
May 6, 2022 (KR) 10-2022-0055999

(51) Int. Cl. G01N 33/574; G01N 33/68; C12Q 1/6886

(52) U.S. Cl. CPC G01N 33/574; G01N 33/6866; C12Q 1/6886; G01N 2800/7028

(57) ABSTRACT

The present disclosure relates to an early cancer diagnosis method and a diagnostic kit using interferon gamma gene concentration measurement in exosomes. By using the interferon gamma gene in exosomes according to the present disclosure, it is possible to early diagnose cancer with high accuracy by a non-invasive method using a liquid biopsy such as blood. In addition, according to the present disclosure, it is possible to supplement the problems of existing tests by quickly and accurately screening and detecting high-risk groups of cancer by a non-invasive method.

### CROSS-REFERENCE TO RELATED APPLICATION

This application claims benefit of Korean Patent Application No. 10-2022-0055999 filed on May 6, 2022. The entire disclosure of the application identified in this paragraph is incorporated herein by reference.

### FIELD

The present disclosure relates to an early cancer diagnosis method and a diagnostic kit using interferon gamma gene concentration measurement in exosomes.

### BACKGROUND

The World Health Organization (WHO) International Agency for Research on Cancer has also reported that the number of deaths from cancer will reach 13.3 million by 2030, nearly doubling the current level. According to the International Agency for Research on Cancer, it is estimated that new cancer patients will surge to 21.3 million by 2030, and 13.3 million of the new cancer cases will die. Compared to 2008 when 12.7 million new cancer patients were newly diagnosed and 7.6 million of these cancer patients died, this is nearly double the number. In particular, in 2008, 56% of new cancer patients and 63% of deaths occurred in developing countries. In Korea, 3 out of 10 people die from cancer. According to a recent survey by the National Statistical Office, 65,000 out of the 246,000 deaths in 2006 died from cancer. This figure corresponds to 26.7% of the total deaths. In particular, the cancer mortality rate is increasing every year. The number of cancer deaths per 100,000 people in 1995 was 110.8, but is increasing rapidly to 122.1 in 2000 and 134.5 in 2005. Currently, cancer is the second leading cause of death among adults in Korea. Considering the economic loss, it is estimated that about 1.3 trillion won is being spent on the treatment of cancer patients, so it is a disease that urgently requires national measures at this point (May, 1999, Chosun Ilbo). As such, since cancer is cured or has a high therapeutic effect when diagnosed early, research on methods for early diagnosis of cancer is being actively conducted in the art. A “cancer metastasis map” was completed by analyzing 7 major cancer patients from 1995 to 2007. This cancer metastasis map is expected to reduce fear of cancer and help predict treatment.

Cancer is a symptom that may not be controlled because normal cells cause abnormalities in growth due to internal/external factors. These abnormal cell growths usually develop into masses of cells known as tumors, infiltrate into surrounding tissues, and then metastasize to other parts of the body. The order of the metastasis rate of the seven major cancers among various cancers is colon cancer (34.7%), gastric cancer (30.1%), lung cancer (28.7%), breast cancer (24.1%), liver cancer (13.1%), cervical cancer (10.3%), and prostate cancer (8.2%), and lung (20.9%), bone (20.7%), and liver (19.7%). The sites where cancer metastasizes the most are lung (20.9%), bone (20.7%), and liver (19.7%). These three organs accounted for 61.4% of the metastases.

On the other hand, cancer diagnosis methods up to now mainly detect the presence of cancer through contrast images such as CT, MRI, and X-rays. There is a problem in that theses tests are mainly performed when a patient has an active will to the tests due to pain or discomfort and it is easy to overlook the existence of cancer because these tests are limitedly performed on specific tissues. A method of determining a possibility of cancer occurrence with blood is also being performed. However, the method uses blood tumor markers for prostate cancer, colon cancer, ovarian cancer, pancreatic cancer, liver cancer, etc, and thus, even if an organ is not cancer, since the method determines the organ as benign when the organ has a pathogenesis, the method still has limitations as a method for diagnosing cancer in fact. Attempts to diagnose cancer using antibodies are also being made, but this is also limited to some cancers. On the other hand, a non-invasive method (blood collection) using blood is very simple and painless, and has the advantage of not requiring hospitalization and recovery period after examination. In addition, such a liquid biopsy has the advantage of enabling early diagnosis even for potential patients who have not developed cancer while avoiding side effects of tissue examination, and periodically monitoring a treatment progress of patients who have already developed cancer.

Exosomes are small membrane vesicles secreted from most cells. It has been reported that various types of proteins, genetic materials (DNA, mRNA, miRNA), lipids, etc., derived from the cells are included within the exosomes. In particular, since there is an excess of RNA-degrading enzyme (RNase) in a serum, it has bee reported that the genetic materials secreted from cells are easily degraded and not easy to measure, but in the case of RNA existing within the exosomes, the RNA is protected from RNA-degrading enzymes and exists stably. As described above, it has been reported that tissue-derived exosomes may be used for diagnosis of diseases because they reflect a state of a tissue that secretes the exosomes.

Meanwhile, interferon gamma (interferon-γ; INF-γ) is known to be very important for the recognition of microorganisms by innate immune cells and the development of cell mediated immunity against intracellular pathogens in an initial host defense. In particular, since T cells sensitive to a specific antigen secrete INF-γ when restimulated by the same antigen, the degree of INF-γ secretion has been applied to the diagnosis of bacterial or viral infection such as mycobacterium tuberculosis and Leishmania braziliensis. Currently, there is a method of measuring the amount of interferon-gamma by the sandwich ELISA method, and there is a method of measuring the concentration (IU/ [symbol] ) of IFNγ in a patient's blood (IU/ml) using an enzyme-linked immunosorbent assay ELISA. However, until now, there is no technology of immediately isolating RNA from whole blood, measuring an expression level of interferon gamma gene using real-time gene amplification technology (qRT-PCR) to measure a concentration of interferon gamma in the blood, and diagnosing cancer using the concentration.

Accordingly, the present disclosure has completed by a method of extracting RNA from blood and plasma, indirectly measuring the expression level of interferon gamma in exosomes present in blood, and screening and early diagnosing high-risk groups of cancer.

### RELATED ART DOCUMENT

### Patent Document

(Patent Document 1) Korean Patent Laid-Open Publication No. 10-2019-0022395

(Patent Document 2) Korean Patent Laid-Open Publication No. 10-2013-0136012

### SUMMARY

The present disclosure provides a composition for diagnosing cancer.

The present disclosure provides a composition for diagnosing cancer.

The present disclosure provides a method of providing information for cancer diagnosis.

The technical problems to be achieved by the present disclosure are not limited to the technical problems mentioned above, and other technical problems that are not mentioned may be clearly understood by those with ordinary knowledge in the technical field to which the present disclosure belongs from the following description.

According to an aspect of the present disclosure, a composition for cancer diagnosis includes a formulation for measuring a concentration of interferon gamma RNA.

The formulation for measuring interferon gamma RNA may be a primer set or a probe that specifically binds to the interferon gamma RNA. The primer set may be a dumbbell structure oligonucleotide primer, and the primer set may include sequence Nos. 1 and 2.

The interferon gamma RNA may be derived from exosomes in blood.

According to another aspect of the present disclosure, a kit for cancer diagnosis includes a formulation measuring a concentration of interferon gamma RNA in blood. The kit may be a real-time gene amplification kit.

According to still another aspect of the present disclosure, there is provided a method of providing information for cancer diagnosis.

The method includes: (a) after removing gDNA from a separated sample, measuring a concentration of interferon gamma RNA; and

(b) when a concentration level of interferon gamma RNA measured decreases in a control group, determining that a patient has cancer or is at a risk of getting cancer.

The interferon gamma RNA of step (a) may be present in exosomes.

### BRIEF DESCRIPTION OF THE DRAWINGS

FIG. 1 is a diagram illustrating results of measuring a concentration according to an expression of interferon gamma gene between a normal group and a patient group using a real-time polymerase chain reaction based on DLP™.

FIG. 2 is a diagram comparing Ct values of a normal group and a cancer patient.

FIG. 3 is a diagram illustrating prediction of various cancers through the measurement of the concentration of the interferon gamma gene in blood and plasma using a real-time polymerase chain reaction based on DLP™ and management guidelines accordingly.

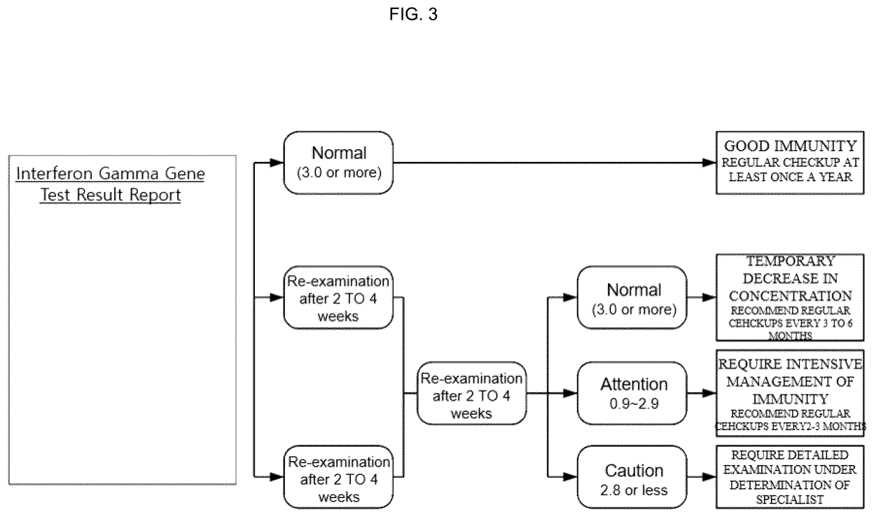

FIGS. 4A and 4B are a diagram a principle of dumbbell structure oligonucleotide of the present disclosure in a target-dependent extension reaction. FIG. 4A illustrates an environment in which amplification may not occur due to high hybridization specificity of the dumbbell structure oligonucleotide under high stringency conditions, and FIG. 4B illustrates that a successful extension reaction of the dumbbell structure oligonucleotide occurs.

### DETAILED DESCRIPTION

The present disclosure provides a composition for cancer diagnosis including a formulation for measuring a concentration of interferon gamma RNA.

Hereinafter, the present disclosure will be described in detail.

The interferon gamma gene may be isolated from a biological sample. As used herein, the term “biological sample” refers to a sample that may be obtained non-invasively, and may be, for example, blood, cells, saliva, sputum, hair, urine, feces, milk, and the like. The interferon gamma gene may be isolated from blood, urine, feces, or milk, but is not limited thereto. As a preferred example, the interferon gamma gene may be isolated from blood. By using a liquid biopsy such as blood, it is possible to diagnose a disease without causing pain to an examiner, unlike an invasive method such as a biopsy. In addition, when using a liquid biopsy, the present invention has a greater advantage compared to a biopsy in performing early diagnosis on a potential patient who has not developed cancer or periodically monitoring a treatment progress of a patient who has already developed cancer.

The concentration of the interferon gamma gene of the present disclosure may be to measure the concentration of RNA expressed by the interferon gamma gene. Preferably, it may be to measure the RNA of exosomes in blood. Generally, since there is an excess of RNA-degrading enzyme (RNase) in a serum, it has been reported that the genetic materials secreted from cells are easily degraded and not easy to measure, but in the case of RNA existing within the exosomes, the RNA is protected from RNA-degrading enzymes and exists stably. As a result, it is possible to measure the RNA in the exosomes by measuring the RNA after removing gDNA from the blood.

In the present disclosure, the formulation for measuring the expression level of the interferon gamma gene may be a primer or a probe that specifically binds to a biomarker according to the present disclosure, but is not limited thereto.

The primer or probe has a sequence complementary to a biomarker sequence of the present disclosure. As used herein, the term “complementary” means having complementarity capable of selectively hybridizing to the above-described nucleotide sequence under certain specific hybridization or annealing conditions. Accordingly, the term “complementary” has a different meaning from the term “perfectly complementary,” and may have one or more mismatched sequences as long as the primer or probe of the present disclosure is capable of selectively hybridizing to the above-described nucleotide sequence.

As used herein, the “probe” refers to a natural or modified monomer or linear oligomer of linkages, and may include deoxyribonucleotides and ribonucleotides, and may hybridize specifically to a target nucleotide sequence and may be naturally occurring or artificially synthesized.

A reporter such as a fluorescent material may be labeled at the end of the probe.

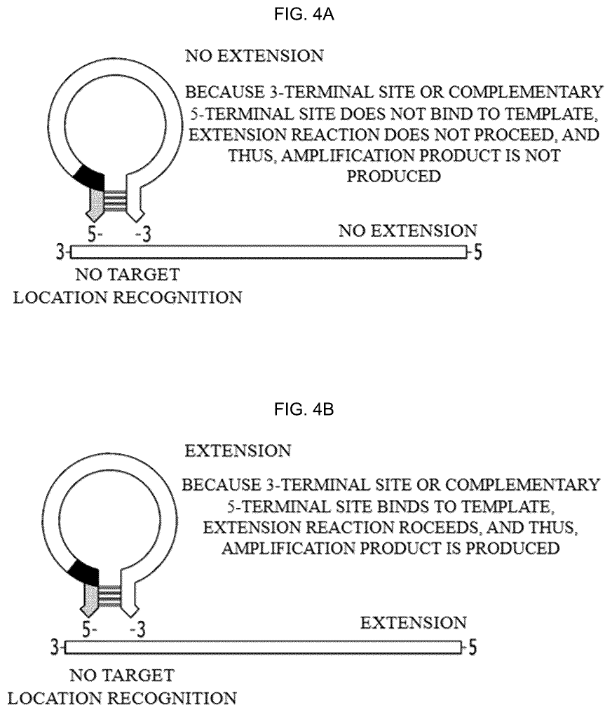

As used herein, the term “primer” means a single-stranded oligonucleotide that may serve as a starting point for the synthesis of a template sequence under suitable conditions (i.e., four different nucleoside triphosphates and a polymerase) in a suitable buffer at a suitable temperature. A suitable length of a primer will vary depending on various factors such as temperature and the application of the primer, but is typically 15 to 30 nucleotides. Short primer molecules generally require lower temperature to form sufficiently stable hybrid complexes with the template. The design of the primer may be easily performed by those skilled in the art with reference to the above-described nucleotide sequence, for example, using a primer design program (e.g., the PRIMER 3 program). However, in the present disclosure, a dumbbell rescue primer set may be used, and the structure and description of the dumbbell structure primer have been described in detail in FIG. 4. In the present disclosure, primer sets of sequence Nos: 1 and 2 capable of targeting interferon gamma RNA may be used.

In the present disclosure, the term “primer” is a nucleic acid sequence having a short free 3′ hydroxyl group, and may refer to a short nucleic acid sequence capable of forming a base pair with a template of a complementary nucleic acid and serving as a starting point for strand copying of a nucleic acid template. The primer may initiate DNA synthesis in the presence of four different nucleoside triphosphates and reagents for polymerization (i.e., DNA polymerase or reverse transcriptase) in an appropriate buffer and temperature. For the primer design, there are various restrictions, such as the A, G, C, and T content ratio of the primer, prevention of primer dimer formation, and prohibition of repeating the same nucleotide sequence three or more times, and the conditions such as the amount of template DNA, the concentration of the primer, the concentration of dNTP, the concentration of Mg2+, the reaction temperature, and the reaction time should be appropriate in the single PCR reaction conditions. The above primers may incorporate additional features that do not change the basic properties. That is, the nucleic acid sequence may be modified using many means known in the art. Examples of such modifications include methylation, encapsulation, substitution of a nucleotide with one or more homologues, and modification of nucleotides into uncharged linkages such as phosphonates, phosphotriesters, phosphoroamidates or carbamates or charged linkages such as phosphorothioates or phosphorodithioates. In addition, the nucleic acid may include one or more additional covalently linked residues of nucleases, toxins, antibodies, signal peptides, proteins such as poly L lysine, intercalating agents such as acridine or psoralen, chelating agents such as metals, radioactive metals, iron oxidizing metals, and alkylating agents. The primer may include a label that may be detected using spectroscopic, photochemical, biochemical, immunochemical, or chemical means. The useful label includes proteins for which 32P, fluorescent dyes, electron-dense reagents, enzymes (commonly used in ELISAs), biotin or haptens, and antisera or monoclonal antibodies are available. The primers of the present disclosure may be chemically synthesized by cloning of an appropriate sequence and restriction enzymolysis and the phosphotriester method of Narang et al. (1979, Meth, Enzymol 68:90-99), the diethylphosphoramidite method of Beaucage et al. (1981, Tetrahedron Lett. 22 1859-1862), and any other well known method including a direct chemical synthesis method such as the solid support method of U.S. Pat. No. 4,458,066.

The “amplification reaction” means a reaction that amplifies a nucleic acid molecule, and may be an amplification method such as multiplex real time-polymerase chain reaction (multiplex RTPCR) as a polymerase chain reaction (PCR), reverse transcription polymerase chain reaction (RT-PCR), ligase chain reaction (LCR), Gap-LCR (WO 90/01069), repair chain reaction (EP 439,182), transcription-mediated amplification (TMA) (WO88/10315), self sustained sequence replication (WO90/06995), selective amplification of target polynucleotide sequences, consensus sequence primed polymerase chain reaction (CP-PCR), arbitrarily primed polymerase chain reaction (AP-PCR), nucleic acid sequence based amplification (NASBA), strand displacement amplification, and loop-mediated isothermal amplification (LAMP), but is not limited thereto. In the amplification method, the PCR may be performed using a PCR reaction mixture including several components known in the art necessary for a PCR reaction. The PCR reaction mixture may contain genomic DNA isolated from a biological sample isolated from a patient to be analyzed and an appropriate amount of DNA polymerase, dNTP, PCR buffer and water (dH2O) in addition to the primer pair provided in the present disclosure.

The PCR buffer is not limited thereto, but may include one or more buffers of Tris-HCl, MgCl2, and KCl. At this time, the MgCl2 concentration greatly affects the specificity and quantity of amplification, and may preferably be used in the range of 1.5 to 2.5 mM. In general, when Mg2+ is excessive, non-specific PCR amplification products increase, and when Mg2+ is insufficient, the yield of PCR products decreases. The PCR buffer may further contain an appropriate amount of Triton X-100 (Triton X-100). In addition, in performing the PCR, the PCR reaction may be performed according to a known method. Although not limited thereto, after predenaturing the template DNA at 94 to 95° C., and then passing through the cycle of denaturation; annealing; and extension, it can be performed in the general PCR reaction conditions for elongation at finally 70 to 75° C., for example, at 72° C. In the above, the denaturation may be carried out at 90 to 99° C., 92 to 97° C., or 94 to 95° C., and the amplification may be performed at 70 to 75° C., 71 to 74° C. or 72° C. The binding temperature may vary depending on the type of primer, for example, it may be carried out at 50 to 59° C., 52 to 57° C. or 55° C. The time and number of cycles of each step may be determined according to conditions generally practiced in the art. Optimal reaction conditions when performing PCR using an SSR primer pair according to an embodiment of the present disclosure are as follows: After predenaturing the template DNA at 95° C. for 5 minutes, it is repeated 30 cycles at 95° C. for 30 seconds, at 55° C. 30 seconds, and at 72° C. for 1 minute, and then finally reacted at 72° C. for 30 minutes.

In the “measurement of expression level,” the DNA of the PCR product may be separated by size according to the method well known in the art. For example, the amplified target sequence may be labeled with a detectable labeling material. In one embodiment, the labeling material may be a material emitting fluorescence, phosphorescence, or radioactivity, but is not limited thereto. Preferably, the labeling material is 6-FAM, NEN, VIC or PET. When the PCR is performed by labeling 6-FAM, NEN, VIC or PET at the 5′-end of the primer during amplification of the target sequence, the target sequence may be labeled with a detectable fluorescent labeling material. In addition, the labeling material may include Cy5 or Cy-3. In addition, in the case of labeling using a radioactive material, when a radioactive isotope such as S is added to a PCR reaction solution during the PCR, the amplification product is synthesized and radioactivity is incorporated into the amplification product, so the amplification product can be radioactively labeled. The primer sets used to amplify the target sequence are as described above.

As used herein, the term “diagnosis” includes determining susceptibility of an object to a particular disease or condition, determining whether an object currently has a particular disease or illness, determining a prognosis of an object afflicted with a particular disease or illness (e.g., identifying a pre-metastatic or metastatic cancer state, determining a stage of cancer, or determining responsiveness of cancer to treatment), and therametrics (e.g., monitoring a condition of an object to provide information on treatment efficacy).

In the present disclosure, the composition of the present disclosure may be used for early diagnosis of cancer. On the other hand, the present disclosure can be used to screen for cancer risk groups (e.g., high risk groups).

As another aspect of the present disclosure, the present disclosure provides a kit for cancer diagnosis including the composition for cancer diagnosis of the present disclosure described above.

The kit of the present disclosure may include a formulation for measuring the expression level of the interferon gamma gene according to the present disclosure, as well as tools or reagents commonly used in the art to be suitable for use as the kit for cancer diagnosis. For example, the tool or reagent may include a suitable carrier, a labeling material capable of generating a detectable signal, chromophores, solubilizing agents, detergents, buffers, stabilizers, and the like. In addition, the kit of the present disclosure may further include a formulation capable of measuring the expression level of a housekeeping gene that may be used as an internal control.

In the present disclosure, the kit of the present disclosure may be a gene amplification kit, but is not limited thereto. The term “amplification” refers to a reaction that amplifies a nucleic acid molecule. Various amplification reactions have been reported in the art, for example, the polymerase chain reaction (PCR) is disclosed in U.S. Pat. Nos. 4,683,195, 4,683,202, 4,800,159.

As another aspect of the present disclosure, the present disclosure includes (a) measuring an expression level of interferon gamma gene in an isolated sample; and (b) when the measured expression level of the interferon gamma gene is reduced compared to a control group, determining that a patient has cancer or is at a risk of getting cancer.

The expression level of the interferon gamma gene may be measured according to a method commonly used in the field of bio kits, and may be measured using, for example, reverse transcriptase polymerase reaction (RT-PCR), competitive reverse transcriptase polymerase reaction (competitive RT-PCR), real-time reverse transcriptase polymerase reaction, an RNase protection assay (RPA), Northern blotting, a gene chip, and the like.

Measuring the expression level of the interferon gamma gene of the present disclosure may confirm the expression level of the interferon gamma gene by measuring the concentration of interferon RNA in the sample. In particular, in order to selectively measure the concentration of the interferon RNA, it is possible to measure the interferon gamma-expressed RNA included in exosomes in the sample by removing gDNA, so the expression level of the interferon gamma gene in the exosomes, that is, the concentration of the RNA may be measured.

As used herein, the term “method for providing information” is the method of providing information on cancer diagnosis, and refers to a method of acquiring informaiton on an onset or likelihood (risk) of onset of cancer.

In the present disclosure, for the cancer diagnosis, when the amount of interferon gamma RNA is 1/3 to 1/2.5 than a normal control group, it may be determined as cancer, and when the amount of interferon gamma RNA is 1/2 to 1/1, it may be determined that the onset likelihood of onset of cancer is high.

### [EXAMPLE 1] INTERFERON GENE EXPRESSION

The interferon gene marker in the blood was obtained, and the standard material was obtained as described in Table 1 for this (Table 1). In order to construct a real-time gene amplification technology using the obtained interferon gamma gene, a dumbbell structure oligonucleotide primer was designed using a computer program for each obtained labeling gene sequence, and each PCR condition was constructed for a positive standard material. A screening test for these labeling was performed on clinical samples using the dumbbell structure oligonucleotide primer constructed in this way.

TABLE 1

| Gene | NCBI | Forward primer (5′→3′) | Reverse primer (5′→3′) |
|---|---|---|---|
| IFNλ | NM_000619.2 | GAGTTXXXXXCATTCAGATGTAGCGG | GAGACXXXXXGACATTCATGTCTTCCT |
| | | ATAATGGAACTC | TGATGGTCTC |
| | | (SEQ ID NO: 1) | (SEQ ID NO: 2) |
| GAPDH | MK476504.1 | GGACAXXXXXCACTAAGCTCGCTTTC | GGATAXXXXXGATGCTCAAGGCCCTTC |
| | | TTGCTGTCC | ATAATATCC |
| | | (SEQ ID NO: 3) | (SEQ ID NO: 4) |

(X in primer sequences in Table 1 is a base of any one of A, T, C, and G)

### [EXAMPLE 2] TOTAL RNA EXTRACTION

Nucleic acids were extracted using the prepared cancer patient plasma and normal control plasma according to the manufacturer's manual. Smart Lab Assist-32 was used as the nucleic acid extraction equipment, and the equipment was warmed up 10 minutes before using the equipment. After removing the vinyl of the enclosed auto plate, the aluminum foil attached to the top surface of the auto plate was removed, and then, 300 μl of the sample was dispensed into each well of the column using a micropipette. Proteinase K stored at 4° C. was taken out and 20 μl of into each well into which the sample was dispensed was dispensed using a micropipette.

An 8-channel strip was mounted on a strip rack frame of a nucleic acid extraction device, the auto plate in which the sample and Proteinase K were dispensed was inserted into the 96-well plate rack of the nucleic acid extraction device, and pushed all the way to the end for mounting, the door was closed, and the program was run according to the sample. After the program of the nucleic acid extraction device was finished, the auto plate was taken out, and the nucleic acids extracted from each well were transferred to a clean microtube. The concentration values of all samples were corrected by measuring the concentration of the extracted total RNA and analyzing the measured concentration.

### [EXAMPLE 3] REMOVAL OF GENOMIC DNA (GDNA)

The gDNA removal and RT procedures were performed on the total RNA extracted using PrimeScript™ RT reagent Kit with gDNA Eraser (product code RR047A, Takara). Specifically, after mixing 2.0 μl of 5×gDNA Eraser Buffer, 1.0 μl of gDNA Eraser, and 7.0 μl of Total RNA, the final volume was adjusted to 10 μl, and the mixed reagent was reacted at 42° C. for 2 minutes, and then stored at 4° C. For analysis, the reagents were mixed in the following proportions, and the reagents were reacted sequentially at 37° C. for 15 minutes and then at 85° C. for 5 seconds, and then stored at 4° C.

TABLE 2

| | | |
|---|---|---|
| 10 | μl | Reaction solution from Step 1 |
| 4 | μl | 5X PrimeScript Buffer 2 |
| 1 | μl | PrimeScript RT Enzyme Mix 1 |
| 1 | μl | RT Primer Mix |
| 4 | μl | RNase Free Dh₂O |
| 20 | μl | Total volume |

### [EXAMPLE 4] INTERNAL CONTROL GENE AMPLIFICATION

Before performing the obtained cancer gene analysis, gDNA was removed from the extracted total RNA and then quantified once again with an internal control primer using the synthesized cDNA of the sample. The reaction conditions were as follows. 4 μl of the synthesized cDNA, 5 μl of qRT-PCR mastermix, and 1 μl of the internal control primer were mixed to complete a final 10 μl mixture, and then the reaction was performed according to the real-time polymerase chain reaction conditions in Table 4 below.

TABLE 3

| | Number of | | | | |
|---|---|---|---|---|---|
| Segment | cycles | Temperature | Time | | Analysis |
| 1 | 1 | 50° C. | 10 | minutes | |
| 2 | 1 | 95° C. | 10 | minutes | |
| 3 | 40 | 95° C. | 20 | seconds | |
| | | 67° C. | 30 | seconds | Fluorescence |

measurement

### [EXAMPLE 5] INTERFERON GAMMA GENE AMPLIFICATION

After 1.0 μl of interferon gamma gene primer, 4.0 μl of template, and 5.0 μl of qRT-PCR master mix for real-time gene amplification were mixed using the template subjected to normalization using the internal control to make a final 10.0 μl of mixed solution, the PCR was performed according to the conditions in Table 5 below.

TABLE 4

| | Number of | | | | |
|---|---|---|---|---|---|
| Segment | cycles | Temperature | Time | | Analysis |
| 1 | 1 | 50° C. | 10 | minutes | |
| 2 | 1 | 95° C. | 10 | minutes | |
| 3 | 40 | 95° C. | 20 | seconds | |
| | | 67° C. | 30 | seconds | Fluorescence |

measurement

At this time, in order to correct the RNA concentration, a house keeping gene (GAPDH) gene was used as a comparison group. To compare the relative amounts of six genes in blood isolated from normal people and cancer patients, RT-qPCR results were analyzed using the following formula.

Fold change=2−ΔΔCt(threshold cycle number)

ΔΔCt=(Ct of gene of IFNG cancer patient serum−Ct of GAPDH cancer patient serum)−(Ct of IFNG normal serum−Ct of GAPDH normal serum)

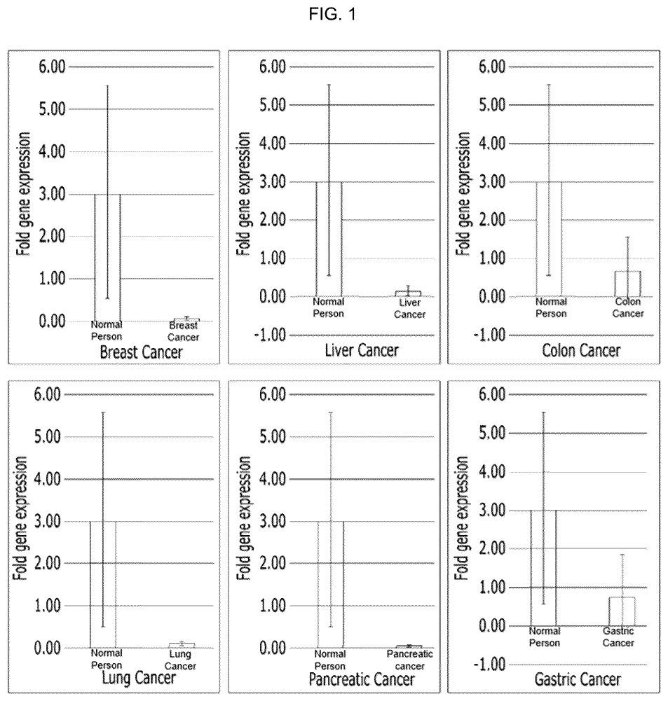

Student's t test was performed with the analyzed results, and a significant difference was determined when p<0.05. The results were shown in FIG. 1.

### Experimental Result

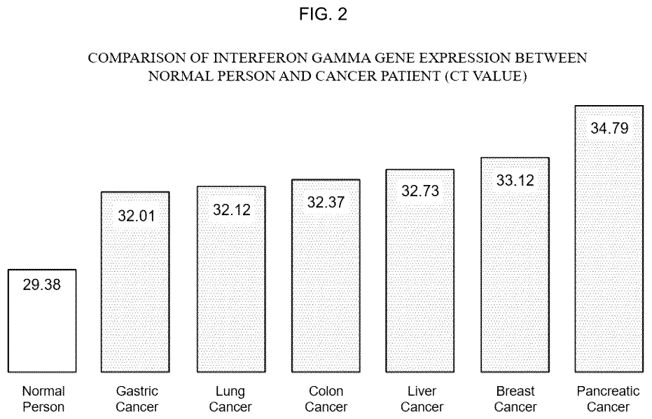

The expression level of the interferon gamma gene, which is expected to be significant in screening the normal and patient groups, was measured. In this case, after the genomic DNA was completely removed from the RNA extracted from the plasma of the normal and patient groups, the internal control (house-keeping gene) was quantified. The results were confirmed in FIG. 2. As a result of confirming the average of Ct values for each normal group and 6 types of cancer using the interferon gamma gene, as shown below, since the interferon gamma gene has a Ct value of 27.30 to 30.78 in the normal group, with an average of 39.38, but has a Ct value of 30.09 to 39.77 in the liver cancer, with an average of 32.73, has a Ct value of 31.53 to 39.83 in colon cancer, with an average of 32.37, and has a Ct value of 29.7 to 34.58 in lung cancer, with an average of 32.12, has a Ct value of 32.14 to 37.22 in breast cancer, with an average of 34.79, has a Ct value of 32.14 to 37.22 in pancreatic cancer, with an average of 34.78, has a Ct value was 25.91 to 34.43 in gastric cancer, with an average of 32.01, significant results were obtained between the 6 types of carcinoma in the normal group and the control group.

In the case of cancer patients, compared to normal people, it could be seen that the expression of IFNë gene was reduced to 16.5 times or more in liver cancer, 23.5 times or more in pancreatic cancer, 9.5 times or more in colon cancer, 10.0 times or more in lung cancer, 6 times or more in gastric cancer, and 3.2 times or more in breast cancer, which is statistically significant. The above results suggest that the IFNG gene in exosomes isolated from the blood of various cancer patients is contained at a lower concentration than in normal people.

In addition, as a result of analyzing the clinical sensitivity and specificity through these clinical specimens, the sensitivity and specificity in gastric and liver cancer were both 91.7%, and the sensitivity and specificity in colon cancer, breast cancer, and pancreatic cancer were both 100.0%, the sensitivity and specificity in lung cancer were 83.4% and 91.7%, respectively, and show a rather low trend, but it was determined that there was no disqualification in early diagnosis of cancer or finding a high-risk group.

By using the interferon gamma gene in exosomes according to the present disclosure, it is possible to early diagnose cancer with high accuracy by a non-invasive method using a liquid biopsy such as blood. In addition, according to the present disclosure, it is possible to supplement the problems of existing tests by quickly and accurately screening and detecting high-risk groups of cancer by a non-invasive method.

It can be understood that the above description of the present disclosure is for illustrative purposes only, and those skilled in the art to which the present disclosure belongs can easily convert the disclosure into another specific form without changing the technical ideas or essential features of the disclosure. Therefore, it is to be understood that the exemplary embodiments described hereinabove are illustrative rather than being restrictive in all aspects.

This application contains references to amino acid sequences and/or nucleic acid sequences which have been submitted concurrently herewith as the sequence listing text file entitled “000030us_SequenceListing.XML”, file size 5.22 kilobytes (KB), created on 14 Nov. 2022. The aforementioned sequence listing is hereby incorporated by reference in its entirety pursuant to 37 C.F.R. § 1.52(e)(5).

What is claimed is:

1. A composition for cancer diagnosis, comprising:
a formulation for measuring a concentration of interferon gamma RNA.

2. The composition of claim 1, wherein the formulation for measuring interferon gamma RNA is a primer set or a probe that specifically binds to the interferon gamma RNA.

3. The composition of claim 1, wherein the interferon gamma RNA is derived from exosomes in blood.

4. The composition of claim 2, wherein the primer set is a dumbbell structure oligonucleotide primer.

5. The composition of claim 4, wherein the primer set includes sequence Nos. 1 and 2.

6. A kit for cancer diagnosis, comprising: the composition of claim 1.

7. The kit of claim 6, wherein the kit is a real-time gene amplification kit.

8. A method of providing information for cancer diagnosis, comprising:
(a) after removing gDNA from an isolated sample, measuring a concentration of interferon gamma RNA; and
(b) when a concentration level of interferon gamma RNA measured decreases in a control group, determining that a patient has cancer or is at a risk of getting cancer.

9. The method of claim 8, wherein the interferon gamma RNA of step (a) is present in exosomes.


SEQUENCE LISTING (4 sequences)

SEQ ID NO: 1 - DNA, 38 bp, unidentified
  source 1..38: mol_type = unassigned DNA
  unsure 6..10
gagttnnnnncattcagatgtagcggataatggaactc

SEQ ID NO: 2 - DNA, 37 bp, unidentified
  source 1..37: mol_type = unassigned DNA
  unsure 6..10
gagacnnnnngacattcatgtcttccttgatggtctc

SEQ ID NO: 3 - DNA, 35 bp, unidentified
  source 1..35: mol_type = unassigned DNA
  unsure 6..10
ggacannnnncactaagctcgctttcttgctgtcc

SEQ ID NO: 4 - DNA, 36 bp, unidentified
  source 1..36: mol_type = unassigned DNA
  unsure 6..10
ggatannnnngatgctcaaggcccttcataatatcc